(19) United States
(12) Patent Application Publication
(10) Pub. No.: US 2025/0352806 A1
(43) Pub. Date: Nov. 20, 2025

(54) EVALUATION SYSTEM AND METHOD FOR EVALUATING AN ESTIMATE OF A PLACEMENT OF IMPLANTABLE ELECTRODE POLES

(71) Applicant: BIOTRONIK SE & Co. KG, Berlin (DE)

(72) Inventors: Michael FRIEDRICH, Kleinmachnow (DE); Gernot KOLBERG, Berlin (DE); Stefan PAULE, Drosendorf (DE); Ingo WEISS, Pforzheim (DE)

(73) Assignee: BIOTRONIK SE & Co. KG, Berlin (DE)

(21) Appl. No.: 18/864,606

(22) PCT Filed: Apr. 26, 2023
(86) PCT No.: PCT/EP2023/060912

§ 371 Date: Nov. 11, 2024

(30) Foreign Application Priority Data
May 25, 2022 (EP) 22175366.8

(51) Int. Cl. A61N 1/372; A61N 1/39

(52) U.S. Cl. CPC A61N 1/372; A61N 1/3956

(57) ABSTRACT

An evaluation system for evaluating an estimate of a placement of implantable electrode poles of an implantable medical device, in particular of a subcutaneous implantable cardioverter defibrillator device, includes an arrangement of electrodes configured to be placed on a patient and a measurement device comprising an excitation circuitry for generating an excitation signal for injection into the patient using said arrangement of electrodes, a sensing circuitry for sensing a sense signal in reaction to said excitation signal using said arrangement of electrodes, and a processing circuitry for processing said sense signal to identify said estimate of the placement of the implantable electrode poles of the implantable medical device.

### CROSS-REFERENCE TO RELATED APPLICATIONS

This application is the United States National Phase under 35 U.S.C. § 371 of PCT International Patent Application No. PCT/EP2023/060912, filed on Apr. 26, 2023, which claims the benefit of European Patent Application No. 22175366.8, filed on May 25, 2022, the disclosures of which are hereby incorporated by reference herein in their entireties.

### TECHNICAL FIELD

The instant invention concerns an evaluation system for evaluating an estimate of a placement of implantable electrode poles of an implantable medical device, and a method for evaluating an estimate of a placement of implantable electrode poles of an implantable medical device.

### BACKGROUND

An implantable medical device such as a subcutaneous implantable cardioverter defibrillator device generally is designed for implantation external to a patient's heart. A subcutaneous implantable cardioverter defibrillator device, in short S-ICD, comprises a generator device having a processing circuitry and a shock generation circuitry, and at least one lead comprising a shock electrode for emitting an electrical shock pulse externally to a patient's heart. The lead is connected to the generator device. The generator device is implanted subcutaneously in a patient. The lead, in a connected state, extends from the generator device, e.g., towards a location in the region of the patient's sternum, the shock electrode hence being placed outside of the patient's heart for emitting an electrical shock pulse at a subcutaneous location external to the patient's heart.

The subcutaneous implantable cardioverter defibrillator device in particular is designed for emitting electrical shocks in case life-threatening arrhythmias of a patient's heart are detected. By means of an electrical shock a defibrillation shall be achieved in order to reset the cardiac rhythm back to a normal state.

When implanting any cardioverter defibrillator device, it is required to place electrode poles of the device within the patient such that the cardioverter defibrillator device reliably may couple energy into the patient's heart in order to achieve a desired action and also reliably may sense cardiac signals. As a prerequisite for implantation, it hence must be identified at which positions to implant the generator device of the cardioverter defibrillator device as well as the shock electrode, such that in operation of the cardioverter defibrillator device shock energy may efficiently couple into the patient's heart in order to achieve a desired action.

With currently available subcutaneous implantable cardioverter defibrillator (S-ICD) systems the generator device (also denoted as can) and a lead carrying a shock electrode are implanted according to anatomical landmarks, and a suitable testing, for example, a threshold testing, is performed after implantation in order to establish whether a defibrillation action may successfully be achieved. Prior to implant, a physician may, for example, temporarily tape the generator device and the lead to the skin of the patient and may try to measure sense signals, which however does not allow for a reliable evaluation of an optimum position of implantation of the generator device and the lead of the cardioverter defibrillator device.

U.S. Pat. No. 10,143,847 describes a method for identifying a position within a patient for a first implantable medical device to be implanted to facilitate tissue conductive communication between the first implantable medical device and a second implantable medical device within the patient.

A.-F. Quast et al., “A novel tool to evaluate the implant position and predict defibrillation success of the subcutaneous implantable cardioverter-defibrillator: PRAETORIAN score”, Heart Rhythm 2019; 16:403-410, describes a score, which is based on clinical and computer modeling knowledge of determinants affecting the defibrillation threshold, for assessing a placement of an electrode of a cardioverter defibrillator device after implantation in a patient.

It is an object of the instant invention to provide an evaluation system and a method which allow to evaluate—prior to implantation of the implantable medical device—a placement of electrode poles of the implantable medical device which is suitable for achieving a therapeutic effect of an electrical stimulation, in particular a subcutaneous implantable cardioverter defibrillator device.

The present disclosure is directed toward overcoming one or more of the above-mentioned problems, though not necessarily limited to embodiments that do.

### SUMMARY

At least this object is achieved by an evaluation system comprising the features of claim 1.

Accordingly, in one aspect, an evaluation system for evaluating an estimate of a placement of implantable electrode poles of an implantable medical device, in particular of a subcutaneous implantable cardioverter defibrillator device, comprises an arrangement of electrodes configured to be placed on a patient and a measurement device. The measurement device comprises an excitation circuitry for generating an excitation signal for injection into the patient using said arrangement of electrodes, a sensing circuitry for sensing a sense signal in reaction to said excitation signal using said arrangement of electrodes, and a processing circuitry for processing said sense signal to identify said estimate of the placement of the implantable electrode poles of the implantable medical device. The processing circuitry is configured to determine a characteristic value indicative of a cardiac motion based on said sense signal and to identify said estimate of the placement based on a comparison of characteristic values of different sense signals obtained, using said arrangement of electrodes, at different locations on the patient.

The evaluation system is to be used prior to implantation of the implantable medical device, in particular the subcutaneous implantable cardioverter defibrillator device. The evaluation system herein shall evaluate at which positions electrode poles of the implantable medical device should optimally be implanted subcutaneously in the patient in order to achieve a desired coupling of the electrode poles to anatomical structures of the patient, in particular the patient's heart, in order to achieve a desired action during subsequent operation of the implantable medical device.

In particular, a subcutaneous implantable cardioverter defibrillator device employs electrode poles in order to generate and emit shock pulses into the patient's heart in order to achieve a defibrillation action of the patient's heart. By means of the evaluation system it shall be established at which positions electrode poles of a subcutaneous implantable cardioverter defibrillator device should optimally be implanted in order to achieve a coupling of the electrode poles to the patient's heart for efficiently allowing to couple shock energy into the patient's heart to achieve a defibrillation action.

Once the estimate of the placement of the electrode poles is determined using the evaluation system, the positions of the electrode poles may be marked on the patient by a user, and at those positions the electrode poles of the implantable medical device should beneficially be implanted in the patient. By means of the evaluation system, hence, prior to implantation an estimate of the placement of the electrode poles may be identified, such that a risk for a poor coupling of the electrode poles to a corresponding anatomical structure is at least reduced.

According to the present invention, the estimated placement of the electrode poles allows the implantable device to achieve a therapeutic effect of an electrical stimulation via the electrode poles. According to an embodiment of the present invention, the estimated placement provides the positions for the electrode poles for an optimum therapeutic effect of an electrical stimulation via the electrode poles.

The evaluation system comprises an arrangement of electrodes comprising multiple electrodes, for example, two, three, four or even far more electrodes, and a measurement device. The measurement device comprises an excitation circuitry for generating an excitation signal, which is injected into the patient by means of the arrangement of electrodes. The measurement device furthermore comprises a sensing circuitry which is configured to sense a sense signal in reaction to the excitation signal using the arrangement of electrodes. A processing circuitry is configured to process the sense signal in order to identify the estimate of the placement of the implantable electrode poles of the implantable medical device, i.e., the locations on the patient at which the electrode poles of the implantable medical device beneficially should be implanted.

The electrodes of the arrangement of electrodes of the evaluation system are generally designed for placement on the patient's skin, such that the electrodes are placed outside of the patient. The electrodes may, for example, employ a contact gel in order to improve an electrical coupling to the patient's skin. The electrodes may be self-adhesive to the patient's skin. The electrodes may be placed on the patient's skin as single electrode elements, or may be placed on a carrier which combines a multiplicity of electrodes for arrangement on the patient.

The processing circuitry is configured to determine a characteristic value indicative of a cardiac motion based on the sense signal and to identify the estimate of the placement based on a comparison of characteristic values of different sense signals obtained, using the arrangement of electrodes, at different locations on the patient. The characteristic value is indicative of a cardiac motion. By means of the characteristic value a measure for the quality of the coupling of the electrodes to the patient's heart may be established. If a signal feature indicative of a cardiac motion is pronounced in the sense signal, this may indicate that the excitation signal couples to cardiac tissue in a pronounced fashion. If the signal feature indicative of a cardiac motion is less pronounced, this allows for the conclusion that the coupling of the excitation signal to cardiac tissue is limited. The characteristic value identifies and quantifies a measure for the signal feature indicative of cardiac motion, such that by comparison of characteristic values of different sense signals a placement of electrodes to establish a desired coupling may be identified.

The identification of the placement generally takes place by comparing characteristic values derived from sense signals obtained using electrodes at different positions on the patient. Based on the comparison of the characteristic values, then, the optimum positions of electrodes may be identified.

In one embodiment, the excitation circuitry is configured to inject the excitation signal into the patient using a first pair of electrodes of the arrangement of electrodes, and the sensing circuitry is configured to sense the sense signal using a second pair of electrodes of the arrangement of electrodes. The first pair of electrodes and the second pair of electrodes generally may use the same electrodes, such that the first pair of electrodes and the second pair of electrodes are equal. In one embodiment, the first pair of electrodes and the second pair of electrodes may use different electrodes, such that the first pair of electrodes and the second pair of electrodes are different in that the first pair of electrodes uses two electrodes which are different than the two electrodes of the second pair of electrodes. In yet another embodiment, the first pair of electrodes and the second pair of electrodes may have one electrode in common, such that only one electrode of each pair differs.

Using the first pair of electrodes the excitation signal is injected into the patient. Using the second pair of electrodes, the sense signal is sensed in reaction to the excitation signal. From the sense signal, then, the characteristic value is derived and compared to characteristic values of other sense signals, such that the estimate of the placement of the electrode poles may be determined.

In one embodiment, the sensing circuitry is configured to sense the different sense signals using the second pair of electrodes at different relative locations of the electrodes of the second pair of electrodes. For example, for sensing the different sense signals the electrodes of the second pair (and beneficially also the electrodes of the first pair for injecting the excitation signal), after a measurement, are placed at different locations to repeat the measurement. The different sense signals hence are sensed in repeated measurements, wherein the processing circuitry of the measurement device compares characteristic values as derived from the different sense signals in order to determine the optimum positions of the electrode poles for implantation of the implantable medical device.

When the electrodes repeatedly are re-positioned at different locations in order to measure sense signals corresponding to different electrode placements in repeated measurements, it may be sufficient if the arrangement of electrodes includes a minimum number of two, three or four electrodes for a 2-pole measurement, a 3-pole measurement or a 4-pole measurement.

In a 2-pole measurement the same electrodes are used for injection of the excitation signal as well as for sensing the sense signal. In a 4-pole measurement different pairs of electrodes are used for injecting the excitation signal and for sensing the sense signal. In a 3-pole measurement one common pole of each pair is used both for injecting the excitation signal and for sensing the sense signal.

However, it also is conceivable that the arrangement of electrodes includes a large number of electrodes. In this embodiment, repeated measurements may be conducted without the need for repeatedly repositioning electrodes at different locations. Rather, the arrangement of electrodes may be used to form a multiplicity of different second pairs of electrodes, wherein the sensing circuitry is configured to sense the different sense signals using the different second pairs of electrodes at different relative locations of the electrodes of the second pairs of electrodes.

In one embodiment, the multiplicity of electrodes may be placed on a carrier to be placed on the patient. The carrier may, for example, be flexibly adaptable to the body shape of the patient. The carrier may, for example, have the shape of an elastic body strap. The carrier may, for example, comprise a velcro closure for easy placement of the carrier on the patient. The carrier may be adhesive in order to ensure a reliable electrical coupling of the electrodes to the patient's skin.

If the arrangement of electrodes includes a multiplicity of electrodes, in particular a multiplicity of first and second pairs of electrodes for injection of excitation signals as well as for sensing sense signals using a variety of different pairs of electrodes, a switching device may be used to switch between the different electrodes to variably form pairs of electrodes for injection of the excitation signal as well as for sensing the sense signal. The electrodes may be arranged on a carrier, for example, in a matrix pattern, wherein by means of the switching device it may be switched between different electrodes for injection of the excitation signal using a first pair at a first relative location and for sensing a sense signal using a second pair of electrodes at a second relative location.

If electrodes are placed on a carrier, the carrier may, for example, comprise a visual indication device for visually indicating electrode locations associated with the placement of the implantable electrode poles of the implantable medical device. Hence, a user is notified, by means of the visual indication device of the carrier, at which locations the electrode poles of the implantable medical device should be implanted, such that a user may, for example, mark the locations on the patient's skin using a pen or the like for the subsequent implantation. The visual indication device may, for example, be a light device, such as an LED. Herein, at each electrode position a light device may be provided, and if a particular electrode position is identified as a suitable implantation site for an electrode pole of the implantable medical device, the visual indication device may provide a corresponding indication.

In one embodiment, the excitation signal is a voltage signal, and the sense signal is a current signal. In one embodiment, the excitation signal is a current signal, and the sense signal is a voltage signal. The excitation signal is fed into the patient's body using a first pair of electrodes of the arrangement of electrodes. The sense signal is received using a second pair of electrodes of the arrangement of electrodes, wherein the first pair and the second pair may use different electrodes or equal electrodes.

In one embodiment, the processing circuitry is configured to derive a measurement signal indicative of an impedance signal from the excitation signal and the sense signal and to determine the characteristic value based on the measurement signal. For deriving the measurement signal, the sense signal and the excitation signal are put in relation to one another, such that a measurement signal indicative of an impedance is determined. The measurement signal may, for example, correspond to an impedance signal or an admittance signal. The measurement signal may, for example, be determined by dividing one signal (the excitation signal or the sense signal) by the other signal (the sense signal or the excitation signal). The measurement signal may in particular vary with time.

The processing circuitry, in one embodiment, is configured to evaluate amplitude information derived from the sense signal. Alternatively or in addition, the processing circuitry may be configured to evaluate phase information derived from the sense signal.

Phase information herein may be evaluated between different sense signals. Alternatively or in addition, phase information may be evaluated between the excitation signal and the sense signal. In one embodiment, the excitation circuitry may be configured to generate excitation signals such that a predefined phase relation between different excitation signals and/or sense signals arises.

In one embodiment, the excitation circuitry is configured to generate the excitation signal in a frequency range between 0.01 Hz to 10 MHz, preferably in a range between 1 kHz to 100 kHz. The excitation signal hence lies in a particular frequency range.

In one embodiment, the processing circuitry may be configured to process the sense signal in a frequency range corresponding to the frequency range of the excitation signal, or in a frequency range smaller than the frequency range of the excitation signal. For example, the processing circuitry may be configured to process one or multiple harmonics of a particular frequency of the sense signal.

In one embodiment, the sensing circuitry uses a lock-in amplifier. Alternatively or in addition, the sensing circuitry may comprise filters, in particular notch filters, for processing certain frequencies of the sense signal.

In one embodiment, the processing circuitry is configured to process predefined time intervals of the sense signal or a signal derived from the sense signal.

In one embodiment, the processing circuitry is configured to determine the characteristic value based on a maximum, a minimum, an integral, and/or a derivative of at least a portion of the sense signal or a signal derived from the sense signal. The characteristic value hence may be determined according to a maximum, a minimum, an integral, and/or a derivative of the sense signal or a signal derived from the sense signal, wherein the maximum, the minimum, the integral and/or the derivative may be determined based on a processing of the sense signal or a signal derived from the sense signal (for example, a measurement signal corresponding to an impedance signal), for example, in the time domain or in the frequency domain.

In one embodiment, the evaluation system comprises at least one of an ECG measurement unit, a Doppler measurement unit, an ultrasound measurement unit, and a sound recording unit. The ECG measurement unit, the Doppler measurement unit, the ultrasound measurement unit and/or the sound recording unit may, for example, be part of the measurement device. In one embodiment, the ECG measurement unit, the Doppler measurement unit, the ultrasound measurement unit and/or the sound recording unit may be a separate unit with respect to the measurement device.

By means of the ECG measurement unit, the Doppler measurement unit, the ultrasound measurement unit and/or the sound recording unit additional information may be obtained which may be used to control the processing of the sense signal as obtained by means of the measurement device in reaction to the excitation signal. For example, timing information may be derived from the ECG measurement unit, the Doppler measurement unit, the ultrasound measurement unit and/or the sound recording unit, for example, to trigger the injection of the excitation signal and/or the sensing of the sense signal and/or the processing of the sense signal.

For example, the processing circuitry may be configured to identify at least a portion of the sense signal or a signal derived from the sense signal based on a physiological event identified using an output of the ECG measurement unit, the Doppler measurement unit, the ultrasound measurement unit and/or the sound recording unit. The processing of signal portions of the sense signal or the signal derived from the sense signal hence is triggered in an event-based manner, based on physiological events as identified using the additional measurement or recording unit. In this way, signal portions of the sense signal or the signal derived from the sense signal, for example, in synchronicity with an ECG signal may be processed.

For example, time intervals in the sense signal or a signal derived from the sense signal may be identified based on an ECG signal, for example, based on an R peak in an ECG signal. For example, a certain time interval may correspond to the systole as identified based on the ECG signal, whereas another time interval may correspond to the diastole as derived from the ECG signal. Alternatively or in addition, time intervals in the sense signal or a signal derived from the sense signal may be identified based on the patient's breathing, as derived, for example, from an output of a sound recording unit or an ultrasound measurement unit. Yet alternatively or in addition, time intervals may be identified based on the closing of a heart valve, for example, by evaluating an output of a sound recording unit. Yet alternatively or in addition, a time interval in the sense signal or a signal derived from the sense signal may be identified to correspond to a signal portion in which the aorta is not filled at a maximum, as, for example, detected using an ultrasound measurement unit.

For example, using the ultrasound measurement unit cardiac motion may be evaluated by employing an ultrasound imaging. Alternatively or in addition, using the Doppler measurement unit blood flow information may be derived.

In one embodiment, the processing circuitry is configured to average signals relating to signal portions of the sense signal or a signal derived from the sense signal. For example, the processing circuitry may be configured to average over signal portions in synchronicity with an ECG signal, each signal portion, for example, relating to a particular interval during the cardiac cycle as identified based on the ECG signal. The averaging may take place over time. Alternatively or in addition, the averaging may employ homologous time points of event-triggered signal portions. For example, a number between 5 to 100 signal portions may be averaged.

The processing of the sense signal or a signal derived from the sense signal by means of the processing circuitry takes place in order to derive a characteristic value. The characteristic value herein is indicative of a cardiac motion, such that by means of the characteristic value that electrode placement may be identified for which a sense signal is obtained in which the cardiac motion is most pronounced, hence indicating a presumably optimum placement of the electrodes for electrical coupling to the anatomy of the patient's heart. This makes use of the assumption that the coupling is optimum for that placement of the electrodes at which a cardiac motion is best identifiable and most pronounced in the sense signal or a signal derived from the sense signal.

For quantifying the characteristic value indicative of the cardiac motion, the sense signal or a signal derived from the sense signal may be processed in the time domain. For example, the characteristic value may be determined as the difference between a maximum and a minimum within a signal portion. In one embodiment, the characteristic value may be determined as the difference between a maximum and a minimum in different signal portions. In one embodiment, the characteristic value may be determined as the standard deviation in a signal portion. In one embodiment, the characteristic value may be determined as the difference between extreme values of signal portions relating to the systole and to the diastole during a cardiac interval. In one embodiment, the characteristic value may be determined as the integral over a signal portion. In one embodiment, the characteristic value may be determined by using a combination of the foregoing. In one embodiment, the characteristic value may be determined as described in the foregoing, but in relation to the absolute average of the impedance, in relation to the maximum or in relation to the minimum of the signal portion.

In one embodiment, for quantifying the characteristic value indicative of the cardiac motion the sense signal may be processed in the frequency domain. For example, to determine the characteristic value the FFT or the spectral power density may be determined. In one embodiment, the amplitude of the spectral line corresponding to the heart rate may be analyzed.

In one embodiment, the processing circuitry may comprise a device for suppressing certain signal portions when determining the characteristic value. The suppression may take place in the time domain by employing a windowing technique. The suppression also may take place in the frequency domain by filtering the sense signal or a signal derived from the sense signal. Signal portions to be suppressed may, for example, stem from a breathing motion of the patient or from artifacts due to motion of the patient. Other disturbing signal portions to be suppressed may relate to electromagnetic disturbances.

In another aspect, a method for evaluating an estimate of a placement of implantable electrode poles of an implantable medical device, in particular of a subcutaneous implantable cardioverter defibrillator device, comprises: providing an arrangement of electrodes for placement on a patient; generating, using an excitation circuitry of a measurement device, an excitation signal for injection into the patient using said arrangement of electrodes; sensing, using a sensing circuitry of said measurement device, a sense signal in reaction to said excitation signal using said arrangement of electrodes; and processing, using a processing circuitry of said measurement device, said sense signal to identify said estimate of the placement of the implantable electrode poles of the implantable medical device. Said processing includes: determining a characteristic value indicative of a cardiac motion based on said sense signal and identifying said estimate of the placement based on a comparison of characteristic values of different sense signals obtained, using said arrangement of electrodes, at different locations on the patient.

The advantages and advantageous embodiments described above for the evaluation system equally apply also to the method.

In one embodiment, the different sense signals are sensed, using the sensing circuitry, in iterative measurements using the arrangement of electrodes. The iterative measurements may be done by repeatedly placing electrodes at different locations on the patient and by then measuring a sense signal in reaction to an injected excitation signal. In one embodiment, the iterative measurements may be done by using an arrangement of a multiplicity of electrodes arranged on a carrier, wherein different electrodes of the multiplicity of electrodes are used in repeated measurements for injecting an excitation signal and measuring a sense signal.

In one embodiment, the estimate of the placement of the implantable electrode poles of the implantable medical device is identified to correspond to the locations of that combination of electrodes for which the largest characteristic value is obtained. The largest characteristic value may correspond to a most pronounced signal feature relating to and being indicative of a cardiac motion, such that by comparing characteristic values associated with different sense signals and by identifying the largest characteristic value the placement of the electrodes and hence the implantable electrode poles may be identified.

A region in which electrodes of the evaluation system may be placed on the patient for the evaluation measurement may be restricted. Boundaries of areas in which electrodes may be placed may, for example, be identified according to anatomical features, for example, a lowest point of the rib cage, the height of the collarbone or the like.

In one embodiment, a minimum number of electrodes for injecting the excitation signal and for sensing the sense signal may be used and manually re-positioned for repeated measurements. For example, two electrodes for a 2-pole measurement, three electrodes for a 3-pole measurement or four electrodes for a 4-pole measurement may be used. The repositioning herein may be controlled by the measurement device, for example, by outputting guidance information to a user via a display of the measurement device.

For example, the repositioning process may take place iteratively starting at an initial positioning of the excitation electrodes and the sense electrodes, wherein from the initial position new positions of the excitation electrodes and/or the sense electrodes may be computed. The repositioning herein may take place by employing an optimization technique, for example, a least squares technique, wherein the iteration ends when a change in an optimization quantity, for example, the characteristic value, from one iteration step to the next iteration step is smaller than a predefined threshold.

The initial positioning herein may be determined according to anatomic features, for example, by measuring a distance to anatomical markers such as the sternum, the collarbone, a certain rib or the like, by employing a medical imaging such as X-ray, CT, MR, ultrasound or optical imaging, or by using ECG measurements.

The iterative repositioning process may employ a triangulation technique by segmenting a region corresponding to the surface of the patient on which the electrodes shall be placed within the evaluation process into triangles, wherein the triangles are successively made smaller during the iteration process.

The electrodes may be tracked using a tracking system, such that the actual positioning of the electrodes is known to the evaluation system at any time. Guidance for using the repositioning to a user may be output via a display, by an optical marking, for example, using a pointer to a body location at which the next electrode should be placed, or by acoustic guidance.

If a multiplicity of electrodes is used, in particular more than 10 electrodes, the electrodes may be placed on a carrier, for example, according to a particular pattern in which the electrodes are arranged equidistantly or non-equidistantly. For example, in certain regions an electrode density may be higher than in other regions, for example, in the right-parasternal or left-parasternal region.

A carrier carrying the multiplicity of electrodes may, for example, have a strap-like shape and may be flexibly bendable and/or elastic. The carrier may comprise a velcro closure for placing and closing the carrier around a patient's body.

Instead of using one single carrier, multiple partial carriers may be employed, which, for example, may be adhesively fixed to a patient or may be fixed to the patient using a bandage.

Yet alternatively, an arrangement of electrodes may be arranged on a stamp device which may be manually or by using a positioning system be pressed against the patient's body.

If a multiplicity of electrodes (more than 10 electrodes) is employed, iterative measurements may be performed by injecting excitation signals and sensing sense signals using different combinations of electrodes. A switching device herein may be provided in order to switch between the different electrodes for providing a pair of excitation electrodes and a pair of sense electrodes.

Once the positioning of the electrodes is determined, the evaluation system may optically indicate the positions at which the electrode poles of the implantable medical device should be implanted. For example, visual indication devices, for example, light devices such as LEDs, on a carrier may indicate an implantation site by lighting up at the corresponding location.

The evaluation system may be configured to output a warning in case no suitable or optimum placement of the implantable electrode poles for the purpose of achieving a successful stimulation therapy of the implantable medical device is identified.

The evaluation of the optimal placement may take place for all electrode poles of the implantable medical device, for some electrode poles or for only one of the electrode poles of the implantable medical device. Herein, one or multiple electrode poles of the implantable medical device may already be implanted, wherein by means of the evaluation system an implantation site for a further electrode pole is identified. The implanted electrode poles of the implantable medical device may herein be used by the evaluation system as electrodes for the injection and/or sensing of signals.

In one embodiment, the evaluation system comprises an assessment unit for assessing the contact quality of the electrodes of the arrangement of electrodes. The assessment unit herein may be configured to output a warning in case the contact quality is below a predefined threshold.

In one embodiment, using the assessment unit the electrodes of the arrangement of electrodes are used for performing tests measurements using a first combination of electrodes. For example, electrodes which are located spatially close together are used for a test measurement in order to determine an impedance value. Then, another, second combination of electrodes is used which are also located spatially close together, but differ from the first combination, for another test measurement in order to determine another impedance value. The impedance values may then be compared. If the impedance values differ by more than a predefined margin, it may be identified that at least one of the combinations of electrodes comprises one or multiple electrodes which are poorly coupled to the patient. The test measurements may be repeated for a multiplicity of different combinations, such that a coupling of substantially all electrodes may be assessed.

The measurement device of the evaluation system may be integrated in a programmer for programming the implantable medical device.

The measurement device may take into account subcutaneous fat when determining the placement of the electrode poles of the implantable medical device. Subcutaneous fat may, for example, be identified using an imaging technique such as ultrasound or impedance tomography.

The evaluation system may be configured to conduct an impedance tomography.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The idea of the present invention shall subsequently be described in more detail with reference to the embodiments as shown in the drawings. Herein:

FIG. 1 shows a schematic drawing of a subcutaneous implantable cardioverter defibrillator device in an implanted state in a patient;

FIG. 2 shows a schematic drawing of an evaluation system for evaluating an estimate of a placement of implantable electrode poles of an implantable medical device;

FIG. 3 shows a carrier carrying a multiplicity of electrodes for placement on a patient;

FIG. 4 shows a schematic drawing indicating regions for placement of electrodes on a patient; and

FIG. 5 shows an example of a sense signal obtained using an evaluation system.

### DETAILED DESCRIPTION

Subsequently, embodiments of the present invention shall be described in detail with reference to the drawings. In the drawings, like reference numerals designate like structural elements.

It is to be noted that the embodiments are not limiting for the present invention, but merely represent illustrative examples.

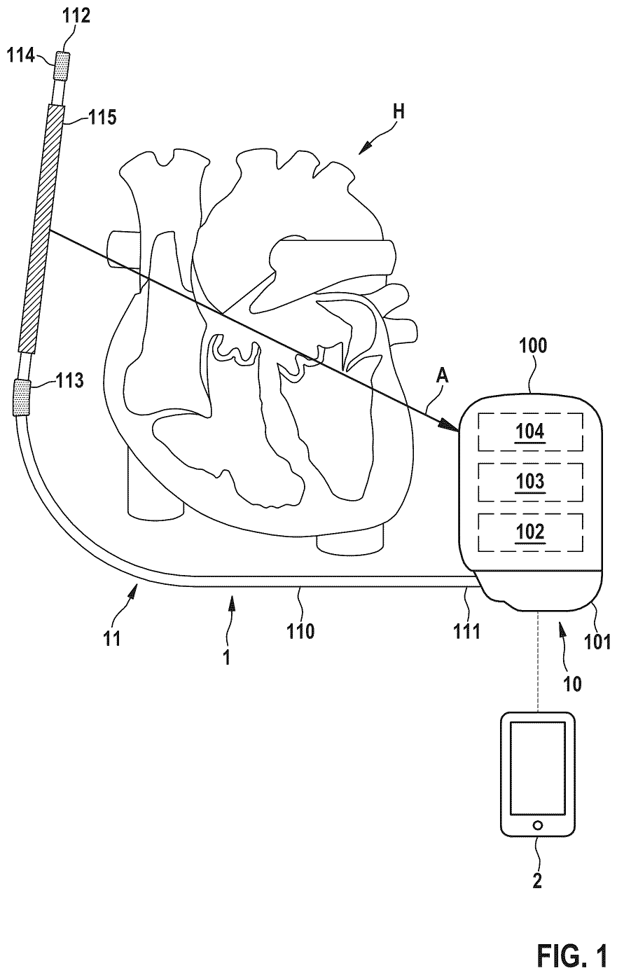

Referring to FIG. 1, in a setup of a therapy system a subcutaneous implantable cardioverter defibrillator device 1 is implanted such that the subcutaneous implantable cardioverter defibrillator device 1 is completely external to the heart H, the subcutaneous implantable cardioverter defibrillator device 1 comprising a generator device 10 encapsulated within a housing 100, and a lead 11 connected to the generator device 10 at a proximal end 111 and carrying electrode poles 113, 114 as well as a shock electrode 115 in the shape of a coil formed on a distal portion close to a distal end 112 of the lead 11.

The electrode poles 113, 114, for example, formed as ring electrodes on either side of the shock electrode 115, serve to sense cardiac signals for processing within the generator device 10 of the subcutaneous implantable cardioverter defibrillator device 1, such that based on sensed signals an arrhythmia may be identified and a shock pulse may be generated for providing for a defibrillation therapy.

The subcutaneous implantable cardioverter defibrillator device 1 is designed for a subcutaneous, non-transvenous implantation, that is an implantation external to the patient's heart H. In particular, the lead 11 connected to the generator device 10 shall extend subcutaneously from the generator device 10, the shock electrode 115 hence, in an implanted state, being placed at a subcutaneous location outside of the heart H for providing for a defibrillation therapy. The generator device 10 likewise is implanted subcutaneously in a patient. The lead 11, with a lead body 110, extends from the generator device 10, for example, towards the sternum of the patient, the lead 11, for example, being placed within subcutaneous tissue in the region of the sternum of the patient.

The subcutaneous implantable cardioverter defibrillator device 1 may comprise a communication interface for communicating with an external device 2, for example, a programmer.

The generator device 10 generally comprises a processing circuitry 102 for controlling operation of the subcutaneous implantable cardioverter defibrillator device 1. In addition, the generator device 10 comprises a shock generation circuitry 103 and an energy storage 104, in particular in the shape of a battery. By means of the shock generation circuitry 103 and controlled by the processing circuitry 102, a shock pulse may be generated and may be emitted across an excitation vector A between the shock electrode 115 on the lead 11 and the generator device 10, the housing 100 of the generator device 10 providing for a counter-electrode to the shock electrode 115 such that the shock electrode 115 and the housing 100 of the generator device 10 together form a pair of electrode poles for achieving a defibrillation action to counteract a defibrillation of the patient's heart H.

By means of the sensing electrodes 113, 114, potentially together with the counter-electrode formed by the housing 100 of the generator device 10, in operation of the implantable cardioverter defibrillator device 1 cardiac signals are sensed and processed by the processing circuitry 102 in order to identify a potential cardiac arrhythmia, such as a fibrillation, in order to control the shock generation circuitry 103 for performing a defibrillation action.

In an implanted state, the shock electrode 115 and the counter-electrode formed by the housing 100 of the generator device 10 shall rest at locations subcutaneously within the patient such that a defibrillation may be achieved efficiently with as little shock energy as possible. The required shock energy herein depends on the coupling of the electrical energy as injected into the patient between the shock electrode 115 and the housing 100 of the generator device 10 to the patient's heart H for causing a transitioning of the patient's heart H, e.g., from a fibrillation state into a normal, rhythmic state. By optimizing the positions of the shock electrode 115 and the counter-electrode formed by the housing 100 of the generator device 10, the coupling of energy into the patient's heart H for achieving a defibrillation action may be improved, and the required shock energy to perform a defibrillation action may be minimized.

Whereas typically a subcutaneous implantable cardioverter defibrillator device 1 as depicted in FIG. 1 is implanted according to anatomical features of the patient, it herein is proposed to use an evaluation system to evaluate, prior to implantation of the subcutaneous implantable cardioverter defibrillator device 1, a placement of the electrode poles 100, 115 of the implantable device 1.

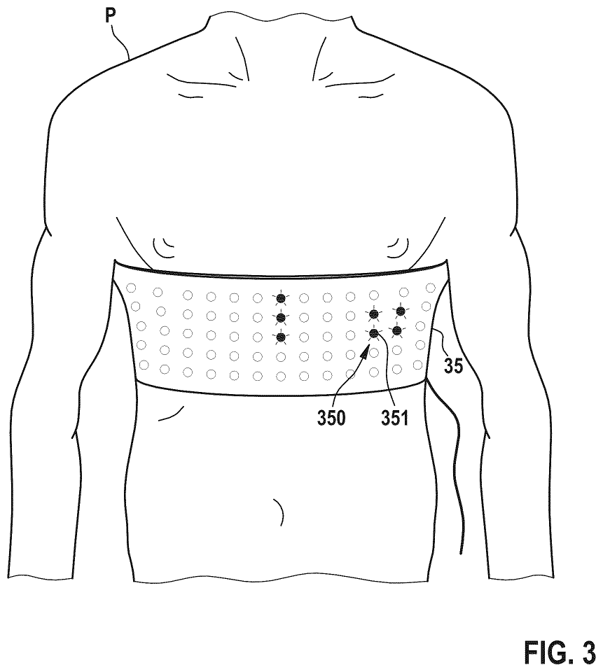

An evaluation system 3 of this kind, in a schematic drawing, is shown in FIG. 3.

The evaluation system 3 comprises a measurement device 30 which is operated external to a patient P and is in connection with an arrangement of electrodes 31-34 which are configured for external placement on the patient's skin. The evaluation system 3 herein may comprise any desired number of electrodes 31-34 (equal to or larger than two). The electrodes 31-34 serve to inject an excitation signal E into the patient P and sense, in response, a sense signal S in order to evaluate, based on the sense signal S and potentially in relation to the excitation signal E, an optimized placement of electrode poles of the implantable cardioverter defibrillator device 1 on the patient P.

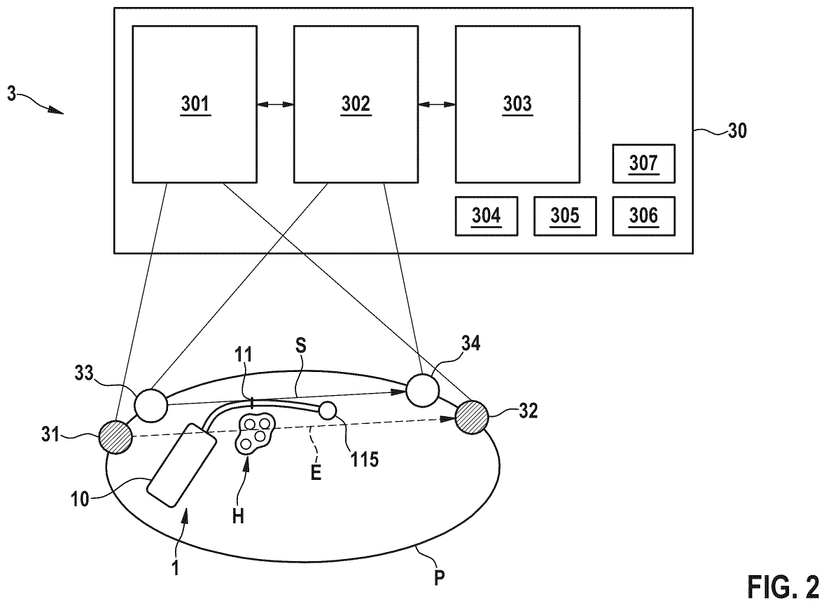

Generally, the evaluation system 3 serves to measure sense signals S in response to excitation signals for combinations of electrodes 31-34 at different locations on the patient P. In the example of FIG. 2, two electrodes 31, 32 form a first pair of electrodes serving to inject the excitation signal E into the patient P. Another, second pair of electrodes 33, 34 serves to measure the sense signal S in response to the excitation signal E. The excitation signal E may, for example, be a voltage signal, in which case the sense signal S is a current signal. Alternatively, the excitation signal E may be a current signal, in which case the sense signal S is a voltage signal.

If, for example, a voltage is applied in between the electrodes 31, 32 serving as excitation electrodes in order to inject the excitation signal E into the patient P, a current as resulting from the excitation signal E is measured using the sense electrodes 33, 34. In this way, for example, a measurement signal indicative of a (time-varying) impedance may be derived by setting the sense signal S and the excitation signal E in relation to one another, such that the resulting impedance for the electrode arrangement is determined.

From the sense signal S or a signal derived from the sense signal S, for example, from a resulting impedance signal, a characteristic value may be determined indicative of a cardiac motion. Based on the characteristic value it may be assessed whether a cardiac feature within the signal is more pronounced or less pronounced, such that it may be determined whether the sense signal S or a signal derived from the sense signal S is impacted by the cardiac motion, which is indicative of a degree of coupling of the excitation signal E to the patient's heart H.

By repeating the measurement for electrodes 31-34 at different locations on the patient P, different characteristic values for different electrode placements may be compared to each other. Based on the comparison, then, that placement of the electrodes 31-34 may be identified at which an optimized coupling to the patient's heart H is obtained. By repeating the measurements and by hence identifying a placement of the electrodes 31-34, an optimum positioning of electrode poles of an implantable medical device, in particular a subcutaneous implantable cardioverter defibrillator device 1, may be identified, the electrode poles of the implantable cardioverter defibrillator device 1 being implanted such that the electrode poles rest subcutaneously at those positions on the patient P which have been identified by the evaluation measurements.

The measurement device 30 comprises an excitation circuitry such 301 configured to generate an excitation signal E for injection into the patient P using a corresponding pair of electrodes 31, 32. The measurement device 30 in addition comprises a sensing circuitry 302 configured to sense, using a pair of sense electrodes 33, 34, a resulting sense signal S. A processing circuitry 303 of the measurement device 30 serves to process the sense signal S in relation to the excitation signal E in order to derive a characteristic value indicative of a quality of effective electrical coupling of the electrode arrangement to the patient's heart H and to conduct an optimization routine to identify the placement of the electrodes 31-34 on the patient P in order to optimize the coupling.

As schematically illustrated in FIG. 2, the measurement device 30 in addition may comprise an ECG measurement unit 304, a Doppler measurement unit 305, an ultrasound measurement unit 306 and/or a sound recording unit 307. By means of the units 304-307 additional measurements may be performed in order to obtain additional information relating to the anatomical structure of the patient P and to states of motion of the patient P and the patient's heart H.

The evaluation system 3 may comprise, in one embodiment, a minimum number of electrodes 31-34, for example, two, three or four electrodes. Using two electrodes, a 2-pole measurement may be conducted, in which the same two electrodes serve as excitation electrodes and in addition as sense electrodes. Using four electrodes, a 4-pole measurement may be conducted, in which two different pairs of electrodes serve as excitation electrodes (31, 32 in FIG. 2) and sense electrodes (33, 34 in FIG. 2). Using three electrodes, a 3-pole measurement may be conducted, in which a pair of excitation electrodes and a pair of sense electrodes have one electrode in common.

If the evaluation system 3 comprises a minimum number of electrodes 31-34, repeated measurements are conducted by repositioning the electrodes in between measurements. The repositioning may be carried out manually by a user, or, for example, by employing a robot arm for automatically repositioning the electrodes 31-34 between measurements.

If the electrodes 31-34 are repositioned manually by a user, the measurement device 30 may provide guidance to the user, for example, by identifying the location at which a particular electrode 31-34 should be placed on the patient, for example, by providing visual guidance on a display or by a visual indication means, for example, using a pointer or the like pointing to the location on which a particular electrode 31-34 should be placed on the patient P.

In one embodiment, the evaluation system 3 comprises a multiplicity of electrodes, in particular more than 10 electrodes, beneficially more than 50 electrodes, for example, more than 100 electrodes, which are arranged on a carrier 35, as it is schematically shown in FIG. 3. The carrier 35 may, for example, be flexibly bendable and may have the shape of an elastic strap which may be placed around the body of the patient P. On the carrier 35, electrodes may be placed at electrode locations 350 according to a particular pattern, such that the electrodes, for example, are equidistantly or non-equidistantly arranged on the carrier 35.

In the embodiment of FIG. 3, the different electrodes at the electrode locations 350 may be connected to a switching device such that different combinations of electrodes may be switched to be used as excitation electrodes and/or sense electrodes, such that repeated measurements may be carried out by employing different electrodes rendered active by the switching device.

Either when using a minimum number of electrodes for repositioning in between measurements or when using a large number of electrodes which are switched in between measurements, the measurement device 30 may run an optimization routine in order to identify the optimum electrode positioning for achieving a beneficial coupling to the patient's heart H. The measurement device 30 in particular may be configured to carry out an optimization routine such as a least squares optimization, using an optimization quantity such as a characteristic value as derived based on the sense signal. Herein, a region of the patient's body may be segmented into triangles at whose corners electrodes for measurements are placed, wherein within the optimization routine the position of the triangles is varied and the size of the triangles is reduced, until an optimum positioning is identified.

Once the optimum positioning of the electrode placement is identified, visual identification devices, such as light devices 351, may light up on the carrier 35 in order to indicate the positions for implantation of the electrode poles 100, 115 of the implantable cardioverter defibrillator device 1, or the measurement device 30 may in another way indicate the optimum positioning on a display or by using a visual aid such as a pointer pointing to the correct locations. A user may mark the positions for implantation, for example, using a pen, on the patient's skin, such that subsequently the implantable cardioverter defibrillator device 1 may be implanted by subcutaneously placing the generator device 10 and the lead 11 with the shock electrode 115 arranged thereon at the identified positions.

The electrodes of the evaluation system 3 may contact the patient's skin using a contact gel for a beneficial electrical coupling. The electrodes may be adhesive such that they self-adhere to the patient's skin.

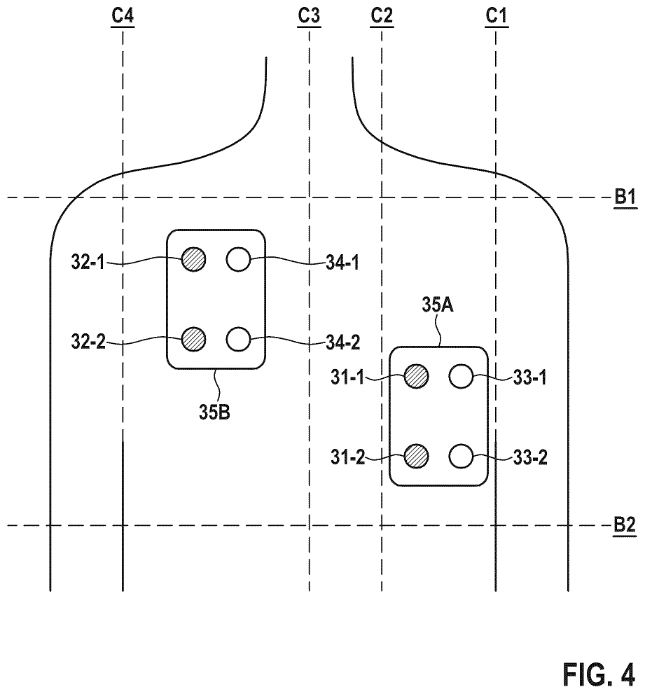

Referring now to FIG. 4, instead of using a single carrier 35, as shown in FIG. 3, electrodes 31-1, 31-2, 32-1, 32-2, 33-1, 33-2, 34-1, 34-2 may be arranged on different carriers 35A, 35B. Herein, regions restricted by boundaries B1, B2, C1, C2, C3, C4 may be identified, for example, in advance, indicating allowable regions for placement of the electrodes 31-1, 31-2, 32-1, 32-2, 33-1, 33-2, 34-1, 34-2. The horizontal boundaries B1, B2 may, for example, relate to a lowest point of the patient's rib cage (boundary B2) and the height of the collarbone (boundaries B1). Vertical boundaries C1-C4 may relate, for example, to anatomical landmarks such as the patient's sternum or the like. The boundaries may be determined by the measurement device 30, for example, using an imaging technique such as an ultrasound measurement unit 306, and may be indicated to a user, for example, on a display or by using a visual aid such as a laser pointing or projection device.

The carrier elements 35A, 35B may be adhesive and may adhere to the patient's skin. Alternatively, the carrier elements 35A, 35B may, for example, be connected to the patient P by an elastic bandage or may be held on the patient P in the manner of a stamp by pressing the carrier elements 35A, 35B to the patient, for example, manually or by using an automated, robotic positioning system.

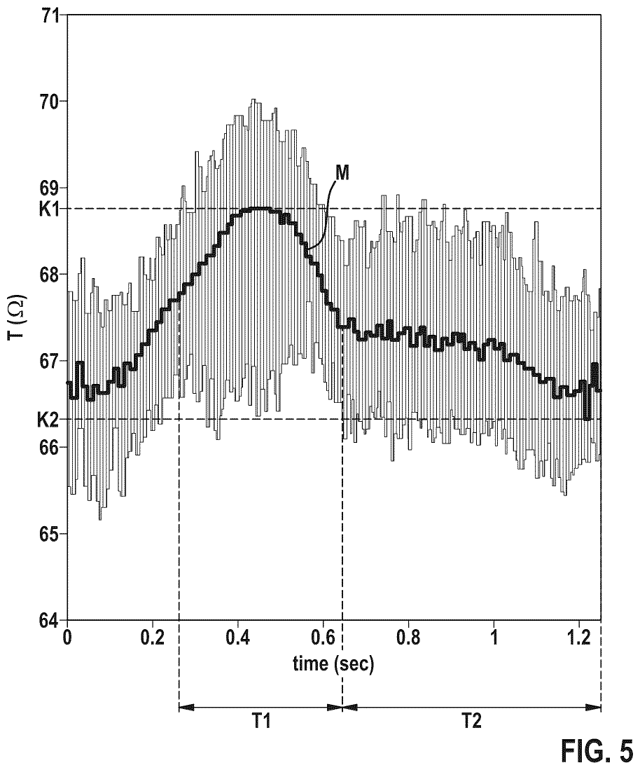

Referring now to FIG. 5, from a measurement signal M as derived from the sense signal S and from the excitation signal E for a particular combination and placement of electrodes a characteristic value may be derived. The measurement signal M may, for example, be an impedance signal determined by the division of a voltage signal by a current signal as injected into respectively sensed from the patient making use of the electrodes. The impedance signal may be modulated by cardiac motion, a characteristic value, for example, corresponding to a maximum K1, a minimum K2, an integral, or a difference between a maximum K1 and a minimum K2 of the impedance signal in one or multiple particular signal portions relating to time intervals T1, T2. In the example of FIG. 5, a characteristic value may, for example, be derived by forming the difference between the maximum K1 in the time interval T1 and the minimum K2 in the time interval T2. Generally, the characteristic value shall be determined such that it is a measure of a signal feature relating to cardiac motion in the measurement signal M, e.g., a large difference between a maximum K1 and a minimum K2 indicating a strong impact on the measurement signal M by cardiac motion and hence a strong coupling to the anatomy of the patient's heart.

The measurement signal M may be obtained by averaging, e.g., over multiple cardiac cycles. Signal portions for averaging herein may be identified, for example, in an event-triggered manner, for example, by employing information obtained from the ECG measurement unit 304, such that signal portions may, for example, be in synchronicity with the patient's heart rate as identified based on an ECG signal as obtained from the ECG measurement unit 304.

By repeated measurements, characteristic values for different placements of electrodes are obtained, such that by comparing characteristic values of different placements a placement may be identified, which then is concluded to be the placement for the electrode poles of the implantable cardioverter defibrillator device 1.

The idea of the present invention is not limited to the embodiments described above.

Further aspects may be generally stated as follows:

In one embodiment, the electrodes consist of an array of at least two electrodes fitted to an elastic belt, where said belt is fastened around the patient's torso such that the electrodes contact the patient's skin at various positions.

In one embodiment, various combinations of the individual electrodes on the patient's skin are connected to the signal generator/evaluation unit either manually or with an automated switching device.

In one embodiment there may be a need, during the position optimization process, to move the electrodes to different locations between two measurements. This can be done manually by the user, whereby the direction and distance of movement (or an ab-solute position) may be instructed by the system. The system can register the electrode position, e.g., with video, ultrasound, linkage mechanism, or other 3D motion tracking method. Electrode repositioning may also be performed automatically, as with a robot arm. In the case of an array of electrodes, no repositioning may be necessary because the system can simply switch on different electrodes in the array. E.g., repositioning may be conducted either manually as instructed by device (gradient method: “go up, left!” to converge upon optimum) or by employing an automatic array switching.

The electrodes preferably offer good electrical coupling to the skin surface, either by virtue of their macroscopic and microscopic surface characteristics, e.g., with a flexible, conforming surface, or by surface finish. Contact can be enhanced by applying electrode gel. Suitable electrode materials may include: Silicone (PDMS) filled with conductive material such as carbon (e.g., in the form of graphite, nanotubes, carbon black) or nickel. Standard ECG electrodes may also be used.

In one embodiment, the system may be used during the subcutaneous ICD implantation procedure, whereby the permanent electronic implant housing (can) is implanted first, then the system is used to find optimal placement of the counter-electrode, e.g., a shock coil.

The positioning optimization system can be integrated in the permanent electronic implant itself.

The positioning optimization system can be integrated in an external programmer that is otherwise used to configure and check the permanent implant system during implantation.

The test electrodes may have the same size and shape as the electrode(s) and/or electronic implant housing (can) of the permanent implant.

In the case of a test electrode array, several adjacent electrodes of the test electrode array can be switched electrically in parallel to create a single effective electrode having a similar shape and/or surface area as the electrode(s) or can to be implanted.

In one embodiment, the system provides a means to indicate the optimal electrode/can positions on the patient's torso (LEDs).

In one embodiment, good electrical contact of the electrodes to the skin may be ensured by using conductive electrode gel on the electrode surfaces, adequate fastening belt tension, inflatable air chambers, suction cups or other suitable means. A mechanical or electromechanical means may be provided to ensure constant contact force between the electrodes and skin; these means may be incorporated in a robotic positioning system.

In one embodiment, the contribution of subcutaneous fat to the overall measured impedance is estimated based on the additional measurement of fat thickness at the electrode sites, using ultrasound transducers or other suitable means.

In one embodiment, the contribution of subcutaneous fat to the overall measured impedance is estimated based on impedance measurements at the electrode sites, using multiple frequencies and thereafter calculating the frequency-dependent impedance caused by fat tissue only.

In one embodiment, the contribution of subcutaneous fat to the overall measured impedance is estimated based on impedance tomography of the distribution of fat within the tissue; this impedance tomography can be performed by the same system

In one embodiment, the contribution of the skin and/or subcutaneous fat to the overall measured impedance is estimated based on the measurement of the local impedance between two or more electrodes in close proximity to one another, for example, between neighboring electrodes in an electrode array.

In one embodiment, the system warns the user if the ICD system to be implanted may not have adequate shock energy to defibrillate, even in the optimal lead/can positions determined by the system.

The system may allow to enhance patient safety by increasing the probability of successful defibrillation with an S-ICD system implanted at these optimized sites. By minimizing the required shock energy in each individual patient, this can reduce the clinically observed wide scatter in DFT values across the entire S-ICD patient population. This in turn reduces the maximum energy the ICD must provide, allowing smaller and less expensive devices (implants with lower voltages, smaller batteries and capacitors) to be used.

The system may provide a means to optimize implant sites before incisions are made, and warn whether the proposed sites may lead to failed defibrillation (i.e., maximum ICD energy will not be sufficient). This increases patient safety and/or may also allow performing S-ICD defibrillation with less expensive, smaller devices.

When a thoracic impedance is measured by sending alternating current between two or more electrodes on the skin of the thorax, there is a slight, periodic fluctuation in the impedance signal amplitude that coincides with the contraction of the heart with each cycle. These fluctuations are caused by changes in heart geometry and the amount of blood in the chambers during each heartbeat. This has been used by some investigators to attempt to detect pulseless electrical activity when no beat-to-beat blood pressure measurement is available; see e.g.,: Alonso et al., “Circulation detection using the electrocardiogram and the thoracic impedance acquired by defibrillation pads”, Resuscitation, 2016 February; 99:56-62.

Next to the impedance measurements, the system can assess whether the sensing signal for these positions is suitable for proper VF detection.

In addition to a coil-can sensing vector, one could also assess other vectors between these and/or other (ring) electrodes along the lead trajectory. In this way, optimal sensing electrode locations (i.e., for choosing the proper lead model/configuration) and/or optimal lead body sub-cutaneous routing (tunneling trajectory) could be determined.

One possible system implementation is an elastic belt fitted with an array of small patch electrodes (perhaps ca. 1×1 cm each). The (reusable) belt is fastened around the patient's torso (perhaps using electrode gel as a coupling aid), and an attached automated device then scans the impedance and sensing signals for all plausible (anatomically realizable) position combinations for shock (coil) electrode, sensing electrodes and can.

The system may be provided beforehand with information on implant geometry, to accommodate different sizes and shapes of the electrical surfaces of the permanent implants to be used. To this end, the system may switch several adjacent test belt electrodes in parallel to create a single effective electrode and therewith simulate the implant's geometry.

Once the optimal positions have been identified, the system can indicate them, for instance by illuminating LEDs at the appropriate locations on the outside surface of the belt. At this point the physician can mark these proposed implant sites on the patient's skin, e.g., with a permanent marker (holes/slits in the belt would be provided for this purpose).

A final check with the actual implants temporarily affixed to the patient's skin (as is done, e.g., with existing S-ICD systems) can be performed to verify that the sensing signals are indeed suitable there.

Alternatively, individual electrodes or small groups of electrodes are on separate patches which can be repositioned manually (under direction of the system) or automatically (e.g., with a robot arm) to various locations during the optimization process.

Proper electrical contact of the electrodes to the skin can be ensured, e.g., by using conductive electrode gel, adequate belt tension, suction cups, and/or by regulating contact pressure, e.g., with a robot arm.

The implanted devices (lead and can) of an S-ICD system are usually inserted below the subcutaneous fat, on the underlying muscle fascia. In order to better simulate these conditions using the test belt on the patient's skin, it may be advantageous to compensate the effects of the skin and subcutaneous fat on the measured impedance.

For instance, fat thickness at the proposed sites could be measured with ultrasound transducers (integrated into the belt or as a separate device), and this value combined with assumed electrical properties of fat is used to estimate its contribution to the overall impedance.

Alternatively, an “impedance signature” of fat (dispersive characteristics, i.e., dependence of conductivity and permittivity on frequency) could be used in multi-frequency measurements to directly identify and calculate out the fat component in the overall signal without knowing its thickness.

Skin conductivity can be measured and compensated, e.g., via impedance measurement from one electrode to its neighbors.

The system could warn if, for this patient, the ICD to be implanted may not have adequate shock energy to defibrillate, even in this optimal position. Conversely, this pre-implantation testing might preclude the need for post-implantation defibrillation testing.

Furthermore, the test system could recommend sites for any additional sensing leads, should the tests indicate that the electrode locations provided by the proposed primary lead and can do not provide suitable sensing vectors.

This feature could also be used post-implantation to propose revisions to the implanted system, in case the patient experiences sensing-related problems, such as inappropriate shocks (e.g., due to T-wave oversensing).

The system can be combined with other devices having extrathoracic electrodes or an electrode array, e.g., a cardiac electroanatomic mapping system, an impedance cardiography/cardiometry device, an impedance tomography device, or an external defibrillator.

An external defibrillator with appropriately modified software could itself be used to find optimal implant placement. In this case one could mask off part of each patch/paddle electrode to match the shock coil/can shape, or use patches already in the correct shape, and manually move the patches to various locations as instructed by the device.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### LIST OF REFERENCE NUMERALS

1 Subcutaneous implantable cardioverter defibrillator device
10 Generator device
100 Housing
101 Connection block
102 Processing circuitry
103 Shock generation circuitry
104 Energy storage (battery)
11 Electrode lead
110 Lead body
111 Proximal end
112 Distal end
113, 114 Electrode pole
115 Shock electrode (coil)
2 External device
3 Evaluation system
30 Measurement device
301 Excitation circuitry
302 Sensing circuitry
303 Processing circuitry
304 ECG measurement unit
305 Doppler measurement unit
306 Ultrasound measurement unit
307 Sound recording unit
31,32 Excitation electrode
31-1, 31-2, 32-1, 32-2 Excitation electrode
33, 34 Sensing electrode
33-1, 33-2, 34-1, 34-2 Sensing electrode
35, 35A, 35B Carrier device
350 Electrode location
351 Indication device (light device)
A Shock vector
B1, B2 Boundary
C1-C4 Boundary
E Excitation signal
H Heart
K1, K2 Characteristic value
M Measurement signal
P Patient
S Sense signal
T1, T2 Portion (time span)

1. An evaluation system for evaluating an estimate of a placement of implantable electrode poles of an implantable medical device, in particular of a subcutaneous implantable cardioverter defibrillator device, comprising;
an arrangement of at least two electrodes configured to be placed on a patient, and
a measurement device comprising an excitation circuitry for generating an excitation signal for injection into the patient using said arrangement of electrodes, a sensing circuitry for sensing a sense signal in reaction to said excitation signal using said arrangement of electrodes, and a processing circuitry for processing said sense signal to identify said estimate of the placement of the implantable electrode poles of the implantable medical device,
wherein the processing circuitry is configured to determine a characteristic value indicative of a cardiac motion based on said sense signal and to identify said estimate of the placement based on a comparison of characteristic values of different sense signals obtained, using said arrangement of electrodes, at different locations on the patient.

2. The evaluation system according to claim 1, wherein said excitation circuitry is configured to inject said excitation signal into the patient using a first pair of electrodes of said arrangement of electrodes, and said sensing circuitry is configured to sense said sense signal using a second pair of electrodes of said arrangement of electrodes.

3. The evaluation system according to claim 2, wherein said sensing circuitry is configured to sense said different sense signals using said second pair of electrodes at different relative locations of the electrodes of the second pair of electrodes.

4. The evaluation system according to claim 2, wherein said arrangement of electrodes, comprises a multiplicity of electrodes including a multiplicity of different second pairs of electrodes, wherein said sensing circuitry is configured to sense said different sense signals using different second pairs of electrodes at different relative locations of the electrodes of the second pairs of electrodes.

5. The evaluation system according to claim 4, wherein said multiplicity of electrodes is arranged on a carrier to be placed on the patient.

6. The evaluation system according to claim 5, wherein said carrier comprises a visual indication device for visually indicating electrode locations associated with said placement of the implantable electrode poles of the implantable medical device.

7. The evaluation system according to claim 1, wherein said excitation signal is a voltage signal and said sense signal is a current signal, or that said excitation signal is a current signal and said sense signal is a voltage signal.

8. The evaluation system according to claim 1, wherein the processing circuitry is configured to derive a measurement signal indicative of an impedance signal from said excitation signal and said sense signal and to determine said characteristic value based on the measurement signal.

9. The evaluation system according to claim 1, wherein said excitation circuitry is configured to generate said excitation signal in a frequency range between 0.01 Hz to 10 MHz, preferably in a range between 1 kHz to 100 KHz.

10. The evaluation system according to claim 1, wherein the processing circuitry is configured to determine said characteristic value based on a maximum, a minimum, an integral, and/or a derivative of at least a portion of said sense signal or a signal derived from said sense signal.

11. The evaluation system according to claim 1, wherein at least one of an ECG measurement unit, a Doppler measurement unit, an ultrasound measurement unit, and a sound recording unit.

12. The evaluation system according to claim 11, wherein the processing circuitry is configured to identify at least one portion of said sense signal or a signal derived from said sense signal based on a physiological event identified using an output of said at least one of a ECG measurement unit, a Doppler measurement unit, an ultrasound measurement unit, and a sound recording unit.

13. A method for evaluating an estimate of a placement of implantable electrode poles of an implantable medical device, in particular of a subcutaneous implantable cardioverter defibrillator device, comprising;
providing an arrangement of at least two electrodes for placement on a patient,
generating, using an excitation circuitry of a measurement device, an excitation signal for injection into the patient using said arrangement of electrodes,
sensing, using a sensing circuitry of said measurement device, a sense signal in reaction to said excitation signal using said arrangement of electrodes, and
processing, using a processing circuitry of said measurement device, said sense signal to identify said estimate of the placement of the implantable electrode poles of the implantable medical device,
wherein said processing includes: determining a characteristic value indicative of a cardiac motion based on said sense signal and identifying said estimate of the placement based on a comparison of characteristic values of different sense signals obtained, using said arrangement of electrodes, at different locations on the patient.

14. The evaluation system according to claim 13, wherein said different sense signals are sensed, using the sensing circuitry, in iterative measurements using said arrangement of electrodes.

15. The evaluation system according to claim 13, wherein the estimate of the placement of the implantable electrode poles of the implantable medical device is identified to correspond to the locations of electrodes at which the largest characteristic value is obtained.
